(19) United States
(12) Patent Application Publication
(10) Pub. No.: US 2006/0004302 A1
(43) Pub. Date: Jan. 5, 2006

(54) Apparatus, systems and methods for diagnosing carpal tunnel syndrome

(75) Inventors: Robert P. Tuckett, Salt Lake City, UT (US); Spencer K. Reese, Taylorsville, UT (US); Richard F. Sesek, Salt Lake City, UT (US); Donald S. Bloswick, Salt Lake City, UT (US)

Correspondence Address: TRASK BRITT, P.O. BOX 2550, SALT LAKE CITY, UT, 84110, US

(73) Assignee: University of Utah Research Foundation, Salt Lake City, UT (US)

(21) Appl. No.: 11/142,572

(22) Filed: Jun. 1, 2005

Related U.S. Application Data
(63) Continuation of application No. PCT/US04/18563, filed on Jun. 10, 2004.
(60) Provisional application No. 60/478,675, filed on Jun. 12, 2003.

(51) Int. Cl. A61B 5/00

(52) U.S. Cl. 600/552

(57) ABSTRACT

Apparatus, systems, and methods for diagnosing carpal tunnel syndrome (“CTS”) are provided. Pressure on the median nerve at the wrist can lead to decreased tactile sensitivity in the fingertips. People with CTS may often experience numbness, tingling, and decreased sensitivity in their finger tips. Compared to a control group, subjects symptomatic of CTS had a greater mean shift (decrease) in tactile sensitivity than the control group when exposed to certain provocations. These provocations include wrist flexion, direct pressure on the transverse carpal ligament area of the wrist, and tendon loading. Additionally, the effects of slight venous occlusion in the forearm were studied. There is an increase in threshold during the recovery period after each provocation. Diagnosis of CTS is provided through monitoring and analysis, preferably with a computer in real-time, of subject's responses to these provocations.

### DECLARATION CLAIMING PRIORITY

This application is a continuation of PCT International Patent Application PCT/US04/018563 filed on Jun. 10, 2004, designating the United States of America, and published in English as WO 2005/000101 on Jan. 6, 2005. Benefit is also claimed from U.S. Provisional Application Ser. No. 60/478,675, filed on Jun. 12, 2003, the contents of each of which are incorporated herein by this reference.

### GOVERNMENT RIGHTS

The United States government may have rights in the following invention pursuant to a grant from the National Institute for Occupational Safety and Health (NIOSH Grant No. T42CCT810426).

### TECHNICAL FIELD

The present invention relates generally to the field of diagnosis of peripheral neuropathy and, more specifically, to the diagnosis of the specific form of peripheral neuropathy known as carpal tunnel syndrome (“CTS”).

### BACKGROUND ART

CTS is caused by compression of the median nerve in the carpal tunnel. It is much more common (three times) in women than in men. It has been attributed to many conditions including anatomical anomalies, fractures, repetitive action, induced trauma, nerve sheath tumors, ganglions, circulatory disturbances, and others. Due to its prevalence in occupations requiring repetitive motion, especially at high force or in awkward wrist postures, CTS is of interest to those studying ergonomics.

If CTS is diagnosed and treated early, permanent damage to the nerve may be avoided. Treatment may include immobilizing the wrist with a splint, discontinuing repetitive motion, using anti-inflammatory medication, and corticosteroid injections. If symptoms continue, the transverse carpal ligament may be sectioned to allow for more space (hence less pressure) within the carpal tunnel.

Several common diagnostic procedures exist. Tapping on transverse carpal ligament (Tinel's sign), placing the wrist in maximal flexion (Phalen's sign), or use of a tourniquet may cause paresthesia within a subject. Direct pressure on the carpal tunnel has also been suggested.

Additionally, nerve conduction studies are often used in the diagnosis of CTS. These require electrically stimulating nerves or muscles and using surface or imbedded electrodes to monitor nerve or muscle response. Conduction velocity is either sensory or motor. Sensory studies seek to find the velocity of conduction of the compound action potential within the nerve, while motor studies seek to measure the recruitment of muscle fibers to a given stimulus (a twitch in the muscle). In diagnosing CTS, sensory studies are preferable.

Although most health care practitioners would define CTS as an entrapment, or compression, of the median nerve at the level of the wrist (i.e., the carpal tunnel), the diagnosis is often not clear cut. A major reason for ambiguity is that in its initial stages, CTS often involves inflammation of the tendons traversing the wrist that control finger movement and grip. As tendonitis progresses, there is a constellation of inflammatory events, including (but not limited to) swelling, vascular stasis, and nociceptor sensitization, which account for many of the clinical signs of CTS. Hence, clinical signs do not clearly differentiate between tendonitis and CTS. Rather, such differentiation requires direct testing of median nerve function specifically localized to the wrist area. At present, only conduction latency across the wrist fulfills these criteria. The alternative of sensory testing (e.g., two point discrimination, monofilament or vibratory threshold) lacks specificity; that is, sensory deficits can be attributed to causes other than CTS.

Of the various types of peripheral neuropathy that afflict the citizens of the United States, CTS has the greatest economic impact. Despite intensive efforts during the past decade to improve detection, treatment and prevention, CTS remains a major and growing problem. Typical assays for carpal tunnel syndrome involve measuring median nerve function (electrophysiological or sensory) with the wrist in a neutral position. Findings of abnormality, as compared to a normal database, in the presence of clinical signs lead to the diagnosis of CTS.

One problem with this approach is that patients often present with clinical signs, but without deficits in median nerve function. Therefore, it remains unknown whether there is early median nerve involvement or merely a case of tendonitis. Since clinical signs can always be simulated, there can remain lingering doubts as to whether a worker or patient might be faking an injury. This state of uncertainty is a stumbling block to effective treatment programs for several reasons: (1) carpal tunnel syndrome, if caught early can be reversed by rehabilitation, ergonomic intervention, and lifestyle counseling; (2) mistrust between management and workers diminishes program effectiveness; (3) at-risk job sites should be identified quickly; and (4) ineffective programs and decision making lead to decreased productivity.

Several forms of peripheral sensory neuropathy exist. Although each can have a major impact on the individual patient, CTS has the greatest impact on the United States as a whole in terms of economic cost as well as patient suffering and disability. For example, in recent years, the total industrial cost of upper extremity repetitive motion injury has approached that of back injury. Nationally, workers compensation costs related to carpal tunnel syndrome are reported to be near $20 billion U.S. dollars annually, with indirect costs to companies estimated to be 4-5 times the direct U.S. dollars spent. The average total cost to industry per carpal tunnel surgery is estimated to be over $30,000 U.S. dollars.

During this decade, there has been a major commitment by the Occupational Health and Safety Administration (“OSHA”) to improve worker safety by lowering the risks of upper extremity cumulative trauma injury. At the present time, there is a major directive from OSHA for establishment of new ergonomic safety regulations and standards that will target upper extremity injury. In addition, the American National Standards Institute (ANSI) is reaching a final consensus on an upper extremity cumulative trauma standard (Z-365) which, if followed by companies, would provide certification of compliance. Surveying for early signs of repetitive motion injury are part of both the OSHA and ANSI initiatives.

In general, the pathophysiological causes of CTS are reversible if caught in the inflammatory stages, i.e., before longer-term fibrotic injury and tissue reorganization have taken place. Hence, there is a need for procedures that can screen for early signs of compression injury so that therapeutic intervention can be implemented before permanent injury has taken place.

It is the contention of OSHA and ANSI that an effective method for reducing cumulative trauma injury is ergonomic change; that is, ergonomically improved design of tools, manufacturing machinery, and workstations would reduce biomechanical stress (i.e., reduce risk factors) and hence reduce frequency of repetitive motion injury. Ergonomic problems are not only expensive to discover and analyze, but it is often even more expensive to implement solutions, not only in terms of skilled manpower, but also capital investment. Hence, it is important to identify at-risk jobs. One method is to identify jobs that have a high percentage of workers with early injuries. Surveillance techniques that identify early injuries help identify at risk jobs that need ergonomic analysis. In addition, measurements of wrist status before and after installation of prototype workstations could help in testing design features before large numbers (or expensive) pieces of equipment are purchased.

Realistically, even with extensive ergonomic investment, there is likely to be a low background level of worker injuries from non-work activities, accidents, previous injuries, and diseases or activities that leave workers predisposed to cumulative trauma (e.g., auto accidents, diabetes, smoking, excessive alcohol consumption). Routine screening programs can help identify injured workers. In addition, studies have shown that it is important to return the injured employee to the work environment as quickly as possible (i.e., return-to-work programs). In such cases physicians must make difficult decisions about whether the employee is able to return to work on a (a) full or (b) part-time basis and whether there should be (c) restricted duty. It is useful for the physician to have available quick and effective means for evaluating the patient's wrist status during the recovery process, not only for (1) therapeutic decision making (i.e., outcomes-based management) but also for (2) reimbursement justification, (3) patient progress reports submitted to the industrial client, and (4) testimony during workers compensation litigation.

Following enactment of the Americans with Disabilities Act, it has become important for companies to give reasonable accommodation to disabled workers (including those previously injured by repetitive motion). Fulfilling this need requires innovative ways of effectively assessing the risk of further injury. Potentially, provocative analysis of carpal tunnel status in disabled workers could help occupational physicians and ergonomic specialists make job choices and worksite modifications that would help the disabled worker be more productive and reduce the likelihood of further injury.

Peripheral neuropathy is a disorder of the peripheral nerves. There are two major measures of sensory peripheral neuropathy: electrophysiological and assays of sensory experience. Electrophysiological techniques involve electrical activation of peripheral axons and then measurement of evoked neuronal activity; for example, (a) compound action potential latency is measured as the time from electrical stimulation until compound action potential wave recording from another point on the peripheral nerve trunk (sensory nerve conduction velocity (sNCV)), (b) muscle twitch latency by placing the electrodes appropriately over the muscle of interest, and (c) central nervous system latency by placing electrodes appropriately on the scalp (somatosensory evoked potential). Also, (d) behavior of individual motor fibers can be evaluated by placing needle electrodes through the skin into the muscle (needle EMG). In each case a unique advantage of electrical activation is the precise timing of the activation is known and hence the conduction time can be evaluated. If there is damage to the peripheral nerve or surrounding structures (e.g., Schwann cells), there is likely to be slowing of conduction. The precise timing of electrical activation allows signal averaging to be used so that small signals can be enhanced by repetitive stimulation. Electrical techniques provide: (1) a direct measure of peripheral nerve status and (2) precise picture of where the stimulation and recording took place (peripheral localization). In addition, (3) electrophysiological techniques were developed long before a detailed knowledge of peripheral receptors was available; and hence, there is a wealth of clinical information is available. (4) No interaction with the patient is required; hence, questions such as malingering and inattention to the test procedure, which accompany psychophysical performance and sensory testing (e.g., hearing, balance, strength, endurance and vision), are not a concern.

Changes in sensory terminal function may occur in early stages of peripheral neuropathy which are not be picked up by traditional electrical procedures. For example, in compression related neuropathies such as CTS, there may be alteration in anterograde and retrograde axon transport which modifies receptor structure and function. In addition, while electrical techniques require supramaximal activation of all rapidly conducting axons to produce a reliable measure of velocity, human microneurography experiments have shown that human subjects can clearly discriminate sensations with activation of single peripheral axons. Hand-held probes for mechanosensory activation range from monofilaments, which measure the smallest “bending force” necessary to produce mechanosensory perception, to two-point discrimination which measures the smallest discernable distance between two probes. More qualitative are tests include lightly touching or rubbing the skin with a cotton wisp. Disadvantages of hand-held probes include (a) lack of precision, (b) randomization, and (c) consistent application of test procedure within and between operators. In addition, it is difficult to guarantee (d) unbiased operation, and (e) the elimination of unconscious cueing between operator and patient, as well as the (f) general problems with psychophysical procedures mentioned above, such as malingering and environmental distractions.

Traditionally, the wrist flexion procedure is defined as by Phalen in which the patient flexes the wrist for a period of 30-60 sec. The development of pain and paraesthesia is consistent with CTS. In 1986, Borg and Lindblom demonstrated that by increasing the duration of flexion up to 15 min, profound changes in median nerve function were produced in patients with electrophysiologically confirmed diagnosis of CTS. More specifically, after 5-8 min delay there was a 230% increase in mechanosensory threshold measured on the pad of the middle finger, which progressed to 470% at 9-12 min and 780% at 13-16 min delay. In control trials, measurement of threshold on the little finger (ulnar distribution) of the same hand showed no significant change in threshold over the same time period. In addition, an age and sex matched patient population with digital paraesthesia, but non-CTS-related conduction velocity abnormalities, showed no significant change in sensory threshold over the same time period of wrist flexion.

As can be determined from the foregoing, a current need exists in the art for improved apparatus, systems, and methods for diagnosis of CTS and for differentiating CTS from other forms of peripheral neuropathy.

### DISCLOSURE OF THE INVENTION

Disclosed are techniques and apparatus used in the techniques that provide evidence of wrist level median nerve entrapment before symptoms become unmistakable by more traditional procedures. The use of this technique and apparatus can specifically diagnose CTS over other forms of peripheral neuropathy.

The technique assesses the effect on nerve function of several provocations applied to the wrist. A provocation is a method for eliciting symptoms of peripheral neuropathy. The technique provides one method of provocation, prolonged wrist flexion, and three additional methods of provocation which enhance the effects on nerve function of prolonged wrist flexion: prolonged wrist flexion with direct pressure on the carpal tunnel region, prolonged wrist flexion with tendon loading on the index and ring fingers, and prolonged wrist flexion with venous occlusion at the forearm. An apparatus used to provide the four methods of provocation is disclosed.

An apparatus for determining whether or not a subject suffers from a peripheral neuropathy includes a stimulation element for applying a sensory stimulation to an area of the subject's body having a nerve, a monitoring element in communication with the stimulating element for measuring the nerve function, and a provocation element that enhances alterations in the nerve's function.

A first diagnostic technique is to establish nerve function for a control group, the control group representing a population asymptomatic for CTS. Nerve function of the control group in the absence of provocation and during a time period when a provocation is applied may be determined. Nerve function of the subject in the absence of provocation and during a time period when a provocation is applied may also be determined. A comparison of the respective nerve functions may indicate whether the subject suffers from a peripheral neuropathy, such as CTS.

A second diagnostic technique is to establish a baseline nerve function of a subject in the absence of provocation. The nerve function of the subject during a time period when a first provocation is applied may be determined. The nerve function of the subject during a time period when an additional provocation is applied may also be determined. A comparison of the respective nerve functions may indicate whether the subject suffers from a peripheral neuropathy, such as CTS.

### BRIEF DESCRIPTION OF DRAWINGS

FIG. 1 is a photograph of the measurement of baseline threshold of a subject according to the invention with the wrist in a neutral position.

FIG. 2 is a photograph of a wrist flexion provocation according to the invention.

FIG. 3 is a photograph of a provocation according to the invention combining wrist flexion with the application of direct pressure to the carpal tunnel region.

FIG. 4 is a photograph of a Durkan Gauge with a fixture according to the invention attached.

FIG. 5 is a photograph of a provocation according to the invention combining wrist flexion with tendon loading.

FIG. 6 is a photograph of a provocation according to the invention combining wrist flexion with venous occlusion.

FIG. 7 is a graph of data obtained from tests according to the invention.

FIG. 8 is a graph of adjusted data obtained from tests according to the invention.

### DETAILED DESCRIPTION OF THE INVENTION

In one embodiment, the present invention uses sensory stimulation, together with provocative means for eliciting symptoms of CTS and monitoring means, to determine whether or not a subject suffers from CTS. The following description illustrates the currently preferred embodiments of the invention.

The mechanosensory threshold, or mechanical sensitivity of a finger may be measured using a computer-controlled vibrometer. The middle or other fingers may be tested. Before the test, a demonstration run may verify for the subject understanding of the procedure. An exemplary method of measuring mechanosensory threshold uses an automated staircase technique. Vibration begins alternatively above, below, or at normal threshold. Stimuli are randomized in times of, by way of example and not to limit the scope of the present invention, between 4 and 7 sec. The subject pushes an event button or provides an alternative signal each time a vibration is sensed. If the subject pushes on the event button at the appropriate time, vibration amplitude on the next trial may be decreased. If the subject pushes the event button (a) outside the appropriate time interval, (b) twice during a stimulus cycle, or (c) does not push the button, the vibration amplitude on the next cycle may be increased. For the first several trials, for example, the vibration amplitude changes in increments to quickly approach the subject's threshold range. One exemplary vibration amplitude change is a 25% increment. Then the vibration amplitude changes in lesser increments, for example, 10% increments. The test ends after the stimulus vibration amplitude has decreased below, and increased above, sensory threshold for at least two complete cycles. The at least two-cycle average of the smallest vibration amplitude sensed is defined as the mechanosensory threshold.

The timing of probe vibration, amplitude, and duration between stimuli may be controlled by a computer. First-order bracketing of threshold can be supplemented with, by way of example, 10 dB or 2 dB steps. A 500 ms vibration may be adequate to provide an unambiguous tactile sensation, and approximately 2 seconds is adequate to allow subjects to decide whether to push the button (normal subjects, ages 18-70 yr.; diabetic patients, ages 24-72 yr.).

The vibrometry procedure is thus used to measure mechanosensory threshold. To measure a baseline mechanosensory threshold, the finger rests on the vibrometry probe with the wrist in a neutral position and the staircase procedure performed as described above. A test run may be completed in about 1.5 min. Multiple measurements of the baseline mechanosensory threshold may alternatively be taken, and a mean value used.

A provocation may then be applied, and a provoked mechanosensory threshold may be determined. Alternatively, the provoked mechanosensory threshold may be determined first, followed by baseline mechanosensory threshold testing. The measurement of provoked mechanosensory threshold is described below.

Sensory nerve conduction latency may additionally be used as a measure of nerve function. One method of determining the sensory nerve conduction of an individual is using electrophysiology techniques. Antidromic sensory nerve conduction may be tested on each of the subjects using surface electrodes. One example of a nerve conduction testing instrument utilizing surface electrodes is marketed under the trade designation Brevio, manufactured by NeuMed, Inc., Pennington, N.J. This nerve conduction instrument reports whether the sensory nerve conduction latency is within the normal range, but requires a 14 cm distance between the active electrode and the stimulator cathode. The active electrode may be placed on a finger, for example, the middle finger. Optionally, the Brevio diagnosis may be omitted. Instead, sensory nerve conduction velocity (sNCV) may be determined. This may be the preferred method on subjects having longer hands.

Several factors can affect conduction velocity including age and temperature. A 2 m/sec per decade over 60 years allowance may optionally be given for subjects over 60 years old. The nerve conduction velocity (V_{measured}) may optionally be corrected for suboptimal skin temperature (31° C. to 34° C.) by using a correlation suggested by DeJesus: V_{corrected}=Vₘₑₐₛᵤᵣₑ·e^{0.0419ΔT}, where ΔT is the difference between the desired skin temperature and the temperature at the time of the measurement and V_{corrected} is the corrected nerve conduction velocity.

The latency must first be measured to determine the sNCV. Recording electrodes may be placed about 7 cm proximal to the wrist over the median nerve, stimulating electrodes may be placed on the “sides” of the finger over the digital nerves, and a ground electrode may be positioned between stimulating and recording sites. Pulses (100 μsec duration, constant voltage isolation, for example) from an electrical stimulator are gradually increased in voltage until a maximum A-β compound action potential wave is recorded from the median nerve. The time from electrical stimulation until the compound wave reaches the median nerve is the latency. Conduction distance from the stimulating electrode to the nearest recording electrode may be approximated by placing a small string on the skin over the approximated nerve path electrode, and then measuring string length with a ruler, for example. Sensory nerve conduction velocity (sNCV) is calculated as distance the signal must travel divided by latency.

Normal, CTS, and non-CTS neuropathy patients may be tested. In addition, for one embodiment, the CTS population may be divided into sNCV positive, faster than normal (+sNCV) and sNCV negative, slower than normal (−sNCV) subpopulations. Experiments have shown 20%-50% of CTS-diagnosed patients to have −sNCV. The subjects determined to be more reactive to prolonged wrist flexion than the control group may be identified as suffering from CTS. +sNCV CTS population is more reactive to sensory threshold changes during wrist flexion than the −sNCV CTS sample. The −sNCV CTS sample is more reactive than normal and non-CTS populations. Hence, vibrometry measurement may be diagnostically valuable in discriminating CTS patients with negative, or inconclusive, electrophysiological workups.

Mechanosensory threshold is not altered by these electrophysiological sensory nerve conduction measurements. Hence, both measurements may optionally be obtained during the same time period. The order of vibrometry procedures and electrophysiology measurements may be randomized for each trial.

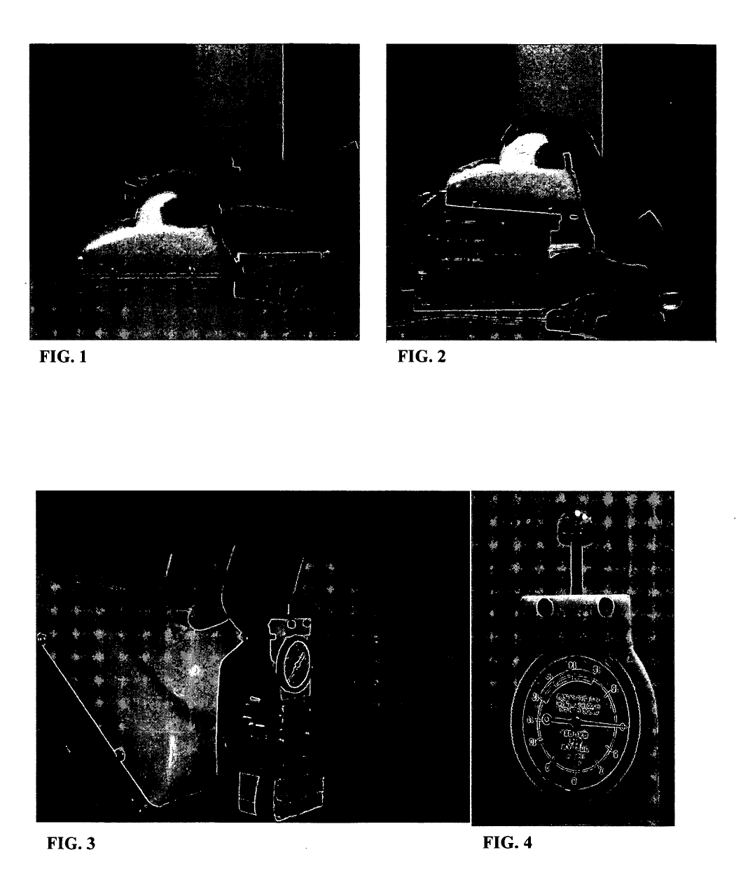

After obtaining baseline values, measurements are repeated during provocative procedures designed to enhance alterations in the median nerve function seen in CTS patients. The testing procedures of the present invention allow a comparison of the effect on nerve function of the basic wrist flexion (Provocation A) with enhancing procedures (Provocations B, C, and D). The nerve function may be tested during multiple separate sessions. Because recovery time post-provocation might be a dependent variable, an interval (rest period) between provocative applications in a given patient may be provided. One exemplary interval is one day. At each session, the baseline nerve function measurements may be found with the wrist in neutral posture (see, FIG. 1).

Mechanosensory threshold and sensory nerve conduction measures may be measured at multiple times during the application of the provocations. A provocation may be applied for an interval, for example, for 15 minutes. The mechanosensory threshold begins to increase, and continues to increase for the duration of the procedure. Up to 15 minutes may be required for dramatic changes in vibratory threshold to occur in CTS patients. Vibrometry measurements may be taken every 2.5 minutes (0, 2.5, 5.0, 7.5, 10.0 12.5, 15.0 minutes). Following the measurement at 15 minutes, the subject may be instructed to massage, shake, or otherwise relive pressure and numbness for one minute. Recovery measurements may then taken at 2.5 minute intervals with the wrist in a neutral posture. These times for test procedures and vibrometry measurements are exemplary, and not limiting.

By way of example, vibrometry measures may require about 1.5 minutes, and the total time between measures may be 2.5 minutes, leaving 1.0 minute to acquire the electrophysiological data. Some threshold measurements may take much longer than 2.5 minutes to run. When this occurs, the next threshold measurement may be skipped so that the following one may be started on time. The sNCV measurement may also be skipped.

During the application of a provocation, care is preferably taken to place the finger in the same position on the vibrometer as in baseline measurement. In addition, the vibrometer is aligned such that the probe travel remains perpendicular to the surface of the skin. The wrist and hand are supported to maintain a constant degree of flexion. The subject may sit on an ergonomic chair so that body position can be adjusted for maximum comfort. The vibrometry procedure and automated staircase technique described above may be used to determine the provoked nerve function, specifically the provoked mechanosensory threshold of the subject.

Subjects may be instructed to maintain a maximum degree wrist flexion [active range of motion (ROM)] during the provocative procedure. Alternatively, a lesser degree of flexion may be used. Verification of flexion angle is obtained by measuring wrist angle with a goniometer at the first, middle and end of the 15 minute test. Additional verification of flexion angle may be obtained.

The first provocation is simply placing the wrist in flexion and maintaining the wrist in that position. The vibrometer may be elevated and the elbow supported by a foam pad (see FIG. 2). Flexion of the wrist increases the pressure within the carpal tunnel, especially in the region of swelling in CTS subjects. The result is decreased sensation within the region of the hand innervated by the median nerve, often in the tip of the middle finger. Hence, wrist flexion may be used as a diagnostic procedure.

The vibrometry procedure and automated staircase technique described above may be used to determine the provoked nerve function, specifically the provoked mechanosensory threshold of the subject under the application of the wrist flexion provocation. The electrophysiology techniques described above may be used to determine sensory nerve conduction of the subject under the application of the wrist flexion provocation.

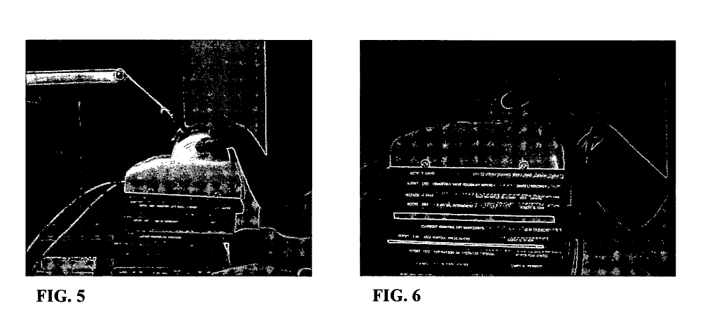

Another provocation combines wrist flexion with the application of pressure directly on the carpal tunnel region (see FIG. 3). In this exemplary procedure, the palmar surface of the wrist in the area of the carpal tunnel is compressed against a rounded (approx. 2 cm diameter), compliant probe connected to a pressure transducer. One instrument that may be used to measurement of the amount of applied pressure is marketed under the trade designation Durkan Gauge (see FIG. 4). A constant gauge reading of, for example, 62 kPa (9 psi) may be maintained throughout the 15 minutes of testing. Alternatively, a compression pad is pressed by the subject against the wrist over the carpal tunnel. A rubber bulb is connected to the compression pad and a pressure manometer is used measure the pressure. The time course of pathophysiology is similar for wrist flexion (Provocation A) and wrist compression procedures, and hence compression may enhance the effects of flexion. The compression pad is pressed Compressive force and wrist flexion are continually maintained within predefined limits. The measurement protocol is as in the vibrometry sections above. Response of individual CTS patients to wrist flexion is compared with and without compression. Wrist compression shortens the time required for wrist flexion to alter mechanosensory threshold in subjects with CTS. These times for test procedures and vibrometry measurements are exemplary, and not limiting.

The third provocation is wrist flexion with tendon loading. Tendon loading affects fingertip sensory deficit. In this test, however, tendon loading may be coupled with wrist flexion. Yet another exemplary embodiment of a provocation involves measuring the mechanosensory threshold and sensory nerve conduction of the subject as in previous experiments for 15 minutes of wrist flexion. During flexion, loops are placed on the middle segment of the index and ring fingers and the tips of the index and ring fingers are pressed against calibrated load cells that are mounted to rigid rods and pre-positioned so that pressure can be exerted without moving the middle finger positioned on the vibrometer (see FIG. 5). Subjects are instructed to generate a pressure level which is estimated to be 50% of maximum effort. The force increases tension on the tendons running through the carpal tunnel to increase the pressure on the median nerve. Force on the load cells is monitored throughout the experiments. These times for test procedures and vibrometry measurements are exemplary, and not limiting. Tension in finger tendons heightens sensory threshold due to direct pressure exerted by the tendons on the median nerve in the carpal tunnel when the wrist is in a flexed position.

The fourth provocation is altered perfusion, or wrist flexion with venous occlusion. Understanding the effect of decreased blood flow on nerves is important, and is thought to be part of the reason that people with CTS experience pain at night. It may be important also in distinguishing between CTS and other peripheral neuropathies such as that caused by diabetes. Occluding venous return from the wrist likely causes stasis, increased capillary pressure, and edema formation in the carpal tunnel region. This combination is thought to enhance the processes that contribute to Phalen and Tinel signs (e.g., pain, paraesthesia, mechanical allodynia) and to blockage of action potentials.

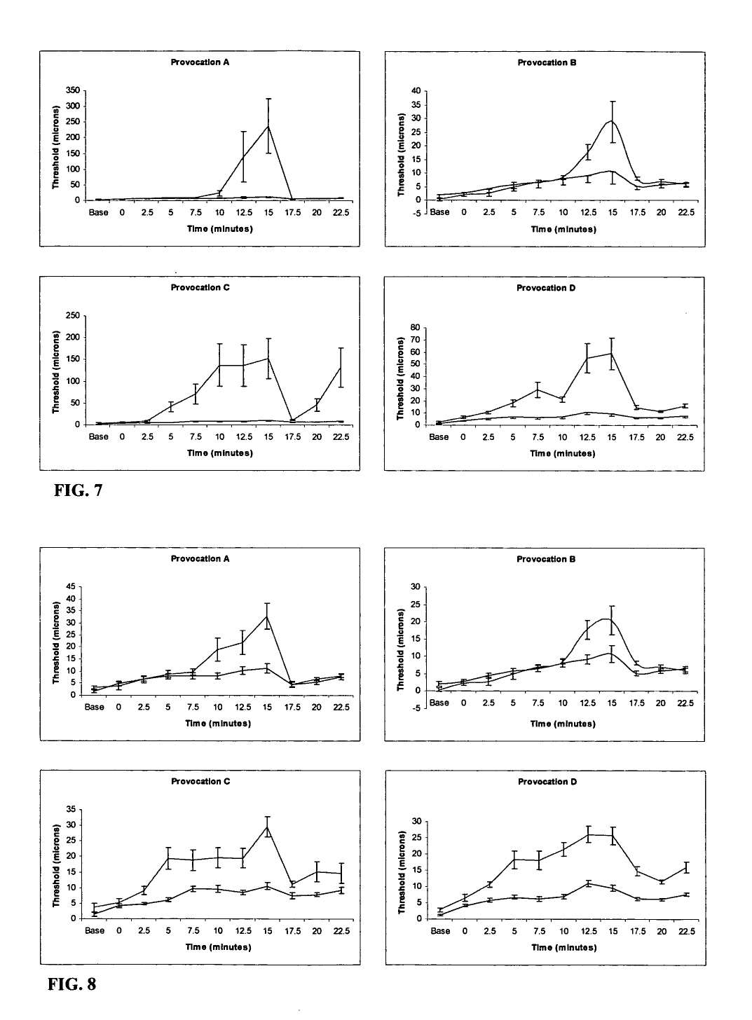

During this exemplary procedure, as depicted in FIG. 6, the wrist is placed in flexion. A pressure cuff is placed loosely around the upper forearm, and the measurement protocol as in the vibrometry sections above is used to obtain a baseline response. After measuring baseline, the pressure cuff is inflated to 2000 Pa (15 mmHg), for example, to restrict venous return from the wrist, causing hypoxia, and perform occlusion in combination with wrist flexion for 15 minute duration. The measurement protocol for mechanosensory threshold and sensory nerve conduction determination may be as above. Response of individual CTS patients to wrist flexion may be compared with and without compression. These times for test procedures and vibrometry measurements are exemplary, and not limiting. Venous occlusion decreases the time required for wrist flexion to alter nerve function in subjects with CTS.

Nerve function data from the four provocations, wrist flexion, wrist compression, venous occlusion, and finger leading each provides a quantification of the enhancement of alterations in the function of the nerve of a subject. Tendon loading has the greatest, occlusion second, and direct pressure the least effect on CTS subjects. It is anticipated that occlusion has a relatively greater effect in diabetic patients.

The presently preferred embodiment of the invention was performed, testing a control group and symptomatic subjects. Subjects were recruited by word of mouth and through flyers posted at medical clinics. Most of the test subjects to date were recruited by word of mouth. The control group consisted of 4 males and 6 females with a mean age of 29 years and a range of 21 to 60 years. Six symptomatic subjects have been recruited with a mean age of 45 and a range of 28 to 62. No subjects were excluded from the study, and none discontinued participation voluntarily. The study was approved by the University of Utah Institutional Review Board (IRB), and subjects read and signed a consent form.

Each subject to be tested was screened, first completing a questionnaire. This requested information regarding current CTS symptoms, risk factors, and related injuries. They were also tested using Phalen's sign (maximum wrist flexion for 60 seconds) and Tinel's sign (gently tapping on the transverse carpal ligament area of the wrist/hand. None of the control subjects tested positive to either Phalen's sign or Tinel's. Four of the symptomatic subjects (67%) tested Phalen's sign positive, while two (33%) tested Tinel's sign positive.

Antidromic sensory nerve conduction latency was tested on each of the subjects using the nerve conduction testing instrument utilizing surface electrodes marketed under the trade designation Brevio and manufactured by NeuMed, Inc., Pennington, N.J. This nerve conduction instrument reports whether the latency is within the normal range, but requires a 14 cm distance between the active electrode (placed on the middle finger) and the stimulator cathode. Since some subjects have longer hands, and therefore a longer distance between the middle finger and median nerve, sensory nerve conduction velocity (sNCV) was found by dividing the distance by the latency. This value was compared to a normal median nerve sNCV of 41.26 m/sec.

Several factors can affect conduction velocity including age and temperature. It is suggested that a 2 m/sec per decade over 60 years allowance be given for subjects over 60 years old. However, the signal amplitude for the only subject over 60 (symptomatic) was not high enough to record the latency, so no age correction was used.

Some of the subjects, despite washing in warm water, had less than the recommended (31° C. to 34° C.) skin temperature. For these people, the nerve conduction velocity was corrected using a correlation suggested by DeJesus: V_{corrected}=Vₘₑₐₛᵤᵣₑ·e^{0.0419ΔT}, where ΔT is the difference between the desired skin temperature and the temperature at the time of the measurement.

Two of the control group subjects had low temperature and low conduction velocities, but exceeded the 41.26 m/sec limit when temperature was corrected to 32° C. The rest of the control group had velocities in the normal range. Two of the six symptomatic subjects exceeded this limit; one without correction, and one correction to 32° C. Thus all control subjects and two symptomatic subjects were sNCV negative.

The test hand for the control group was the least symptomatic (or non-dominant if both were equally asymptomatic). For the symptomatic group, the most symptomatic (or dominant if both were equally symptomatic) hand was chosen unless there previous injuries unrelated to CTS.

Vibrotactile studies were used to determine mechanosensory threshold. Mechanical sensitivity of the middle finger is measured using a computer-controlled vibrometer. The timing of probe vibration, amplitude, and duration between stimuli (50 Hz) were controlled by the computer. The subject pressed a button when a stimulus was sensed. The amplitude was decreased to find the smallest vibration sensed. This smallest vibration is the mechanosensory threshold.

The mechanosensory threshold was tested during four separate sessions with at least 24 hours between visits. At each session, a baseline mechanosensory threshold was found with the wrist in neutral posture (see FIG. 1). Then the mechanosensory threshold measurement was begun at the start of flexion (time 0) and at each 2.5 minute interval while the wrist was placed in one of four provocations for 15 minutes. Following the measurement at 15 minutes, the subject was instructed to massage, shake, or otherwise relive pressure and numbness for one minute. Three recovery measurements were then taken at 2.5 minute intervals with the wrist in a neutral posture.

The four provocations, presented to the subject in randomized order, were A) wrist flexion (FIG. 2), B) wrist flexion with direct pressure on the carpal tunnel (FIG. 3), C) wrist flexion with tendon loading (FIG. 5), and D) wrist flexion with venous occlusion (FIG. 6).

One of the provocations, provocation A, was simply placing the wrist in flexion. The vibrometer was elevated and the elbow was supported by a foam pad (see FIG. 2). Flexion of the wrist increases the pressure within the carpal tunnel, especially in the region of swelling in CTS subjects. The result is decreased sensation within the region of the hand innervated by the median nerve, often in the tip of the middle finger. Hence, wrist flexion may be used as a diagnostic procedure.

Another provocation, provocation B, combined wrist flexion with the application of pressure directly on the carpal tunnel region (see FIG. 3). Pressure was applied with a rounded (approx. 2 cm diameter) probe on a Durkan Gauge (Gorge Medical; Hood River, Oreg.; see FIG. 4). A gauge reading of 62 kPa (9 psi) was maintained throughout the 15 minute of testing. A 62 kPa (9 psi) gauge reading was found to correspond to about 16.8 N (3.8 lbf) Direct pressure is hypothesized to increase the interstitial pressure on the median nerve above that of flexion alone, much as edema.

Yet another provocation, provocation C, combined wrist flexion with tendon loading. Tendon loading alone has an effect on fingertip sensory deficit. In this test, however, tendon loading was coupled with wrist flexion. Loops were placed on the middle segment of the index and ring fingers. These loops were connected by a system of strings and pulleys to weights (see FIG. 5). The force was intended to increase tension on the tendons running through the carpal tunnel to increase the pressure on the median nerve.

The final provocation, provocation D, combined wrist flexion with venous occlusion. Understanding the effect of decreased blood flow on nerves is important, and is thought to be part of the reason that people with CTS experience pain at night. It may be important also in distinguishing between CTS and other peripheral neuropathies such as that caused by diabetes. In this test, the wrist was place in flexion as before and a pressure cuff was placed on the forearm. The pressure was raised to 2000 Pa (15 mm Hg) to slightly occlude the veins (see FIG. 6), causing hypoxia.

FIG. 7 shows the mechanosensory threshold vs. time for each provocation. The Symptomatic group is represented by the top line. Error bars represent the standard error of the mean at each time. A generally increasing trend was seen among both groups during the fifteen minutes of provocation. This was followed by a reduction at the first recovery point then another general increase in threshold over the last two recovery points.

None of the control group mechanosensory thresholds exceeded 39 μm on any test. However, several in the symptomatic group exceeded the limit of the machine (over 600 μm). This contributed to large variance in the symptomatic group data. While this prevents demonstrating statistical significance, it shows the expected trend.

FIG. 8 shows the adjusted data such that mechanosensory thresholds above 50 μm were set equal to 50 μm. This accounts for 21 observations, all among symptomatic subjects. The data plots show the same trend, but the variance in the symptomatic group is reduced.

Some mechanosensory threshold measurements may take much longer than 2.5 minutes to run. When this occurs, the next measurement may be skipped so that the following one may be started on time. This occurred twice at the 12.5 minute period among symptomatic subjects (with particularly high thresholds) during Test B, and the next measurement was skipped. Exclusion of these points caused the mean threshold to drop drastically between the 10 and 15 min means, so a valley appeared on the plot at 12.5 minutes. Because of this, the Test B data for these two subjects were excluded from the plots.

The data were compared to see if the differences in the means at each time were significant. Repeated measures using analysis of variance (ANOVA) showed that there was significant difference in the normal data (for provocations A, C, and D p<0.0001, for B p=0.0036). The symptomatic data were compared using a nonparametric ANOVA because of the significant differences in variance among the groups. The difference between times in each test was once again significant (p<0.03 for each). The significance of the data is designated by the p-value.

Further, for each test, the mean mechanosensory threshold at each time was compared to the baseline. The values of these comparisons are shown in Tables 1-4. Then the mean mechanosensory threshold at each time was compared to the threshold for the previous time. Because all groups of data (for each test at each time within each study group) passed normality tests, paired t-tests were performed when making comparisons within study groups. SEM is the standard error of the mean.

TABLE 1
Threshold relative to baseline: Flexion.

| | Control Group | | Symptomatic Group | |
|---|---|---|---|---|
| Time | Difference | | Difference | |
| (min) | (μm ± SEM) | P - Value | (μm ± SEM) | p - Value |
| 0.0 | 3.19 ± 1.11 | 0.0184 | 0.7167 ± 1.44 | 0.6404 |
| 2.5 | 5.22 ± 0.71 | <0.0001 | 3.45 ± 1.51 | 0.0709 |
| 5.0 | 6.3 ± 0.85 | <0.0001 | 5.6 ± 1.31 | 0.0078 |
| 7.5 | 6.44 ± 1.02 | 0.0001 | 6.55 ± 1.62 | 0.01 |
| 10.0 | 6.58 ± 1.49 | 0.0017 | 20.95 ± 11.29 | 0.1226 |
| 12.5 | 8.67 ± 1.65 | 0.0005 | 136.78 ± 124.16 | 0.3324 |
| 15.0 | 9.71 ± 2.23 | 0.0018 | 235.75 ± 126.55 | 0.1215 |
| 17.5 | 2.93 ± 1.09 | 0.0251 | 1.417 ± 1.93 | 0.4959 |
| 20.0 | 3.86 ± 0.96 | 0.003 | 3.6 ± 1.42 | 0.0519 |
| 22.5 | 6.08 ± 1.4 | 0.0019 | 4.75 ± 1.12 | 0.0082 |

Comparing the mean mechanosensory threshold at each time to the baseline showed significant difference at all times for each test except for test B at 0 (p=0.2889), 2.5 (p=0.0969), 7.5 (p=0.051), and 12.5 minutes (p=0.1048) for the control group. In the symptomatic group, few comparisons were significant.

TABLE 2
Threshold relative to baseline: Flexion and direct pressure.

| | Control Group | | Symptomatic Group | |
|---|---|---|---|---|
| Time | Difference | | Difference | |
| (min) | (μm ± SEM) | p - Value | (μm ± SEM) | p - Value |
| 0.0 | 0.74 ± 0.66 | 0.2889 | 3.75 ± 3.82 | 0.3709 |
| 2.5 | 2.37 ± 1.28 | 0.0969 | 3.317 ± 4.35 | 0.4776 |
| 5.0 | 3.72 ± 1.01 | 0.005 | 7.333 ± 4.88 | 0.1932 |
| 7.5 | 3.35 ± 1.49 | 0.051 | 13.367 ± 6.07 | 0.0787 |
| 10.0 | 6.05 ± 1.47 | 0.0026 | 123.6 ± 101.66 | 0.2783 |
| 12.5 | 4.29 ± 2.38 | 0.1048 | 17.375 ± 6.54 | 0.0743 |
| 15.0 | 8.65 ± 3.5 | 0.0356 | 132.82 ± 99.88 | 0.241 |
| 17.5 | 3.11 ± 0.72 | 0.002 | 8.35 ± 2.52 | 0.0211 |
| 20.0 | 3.85 ± 0.85 | 0.0014 | 5.833 ± 1.53 | 0.0124 |
| 22.5 | 4.36 ± 1.28 | 0.0077 | 6.4 ± 2.67 | 0.062 |

TABLE 3
Threshold relative to baseline: Flexion and tendon loading.

| | Control Group | | Symptomatic Group | |
|---|---|---|---|---|
| Time | Difference | | Difference | |
| (min) | (μm ± SEM) | p - Value | (μm ± SEM) | p - Value |
| 0.0 | 2.77 ± 0.95 | 0.0168 | 1.52 ± 1.51 | 0.3716 |
| 2.5 | 3.44 ± 1.09 | 0.0118 | 5.34 ± 2.43 | 0.093 |
| 5.0 | 4.7 ± 0.67 | <0.0001 | 38.2 ± 27.74 | 0.2405 |
| 7.5 | 8.18 ± 1.84 | 0.0016 | 67.38 ± 57.54 | 0.3066 |
| 10.0 | 8.14 ± 2.07 | 0.0035 | 133.4 ± 122.57 | 0.3376 |
| 12.5 | 7.09 ± 1.56 | 0.0014 | 133.28 ± 122.59 | 0.3381 |
| 15.0 | 9.15 ± 2.01 | 0.0014 | 148.98 ± 119.16 | 0.2793 |
| 17.5 | 6.04 ± 1.4 | 0.0019 | 7.58 ± 0.4 | <0.0001 |
| 20.0 | 6.38 ± 0.6 | <0.0001 | 41.84 ± 36.52 | 0.3159 |
| 22.5 | 7.83 ± 1.54 | 0.0007 | 128.58 ± 123.76 | 0.3575 |

TABLE 4
Threshold relative to baseline: Flexion and venous occlusion.

| | Control Group | | Symptomatic Group | |
|---|---|---|---|---|
| Time | Difference | | Difference | |
| (min) | (μm ± SEM) | p - Value | (μm ± SEM) | p - Value |
| 0.0 | 2.99 ± 0.62 | 0.001 | 3.76 ± 3.73 | 0.3705 |
| 2.5 | 3.60 ± 1.37 | 0.0271 | 7.88 ± 3.36 | 0.0791 |
| 5.0 | 5.57 ± 1.11 | 0.0007 | 15.46 ± 8.53 | 0.1442 |
| 7.5 | 5.11 ± 1.35 | 0.0043 | 26.28 ± 19.44 | 0.2478 |
| 10.0 | 5.84 ± 1.48 | 0.0033 | 18.58 ± 6.56 | 0.0472 |
| 12.5 | 8.28 ± 2.58 | 0.0107 | 52.42 ± 35.84 | 0.2174 |
| 15.0 | 8.45 ± 2.19 | 0.0039 | 56.06 ± 40.08 | 0.2344 |
| 17.5 | 5.15 ± 0.84 | 0.0002 | 12.02 ± 3.73 | 0.0323 |
| 20.0 | 5.00 ± 0.59 | <0.0001 | 8.78 ± 2.61 | 0.0281 |
| 22.5 | 6.53 ± 1.04 | 0.0001 | 13.28 ± 5.89 | 0.0872 |

When mean thresholds at each time were compared to the threshold at the time before, significant difference was seen in provocation A between 0 and 2.5 minutes and 15 and 17.5 minutes and in provocation C between 5 and 7.5 five minutes for the control group. For the symptomatic group, significant difference was seen in provocation C between 0 and 2.5 minutes and in provocation D between 0 and 2.5 minutes.

Between each test (provocation), the mean thresholds were compared at each time. Significant difference was seen between provocations A and C at 2.5 and 20 minutes, and between B and C at 7.5 minutes. This does not statistically demonstrate that adding other risk factors to flexion causes a substantial change in the effect on the median nerve.

Control data for each provocation were compared to symptomatic data at each time interval. Comparisons were made using unpaired t-tests, allowing for unequal variance. The results are tabulated in Table 5. Though there are large differences between the groups at many of the time intervals during provocation, statistical tests do not show significance in these differences. This may also be attributed to the large variance in symptomatic data.

TABLE 5
Threshold Comparisons: Symptomatic minus control

| | Flexion | | Flexion + Direct Pressure | | Flexion + Tendon Loading | | Flexion + Venous Occlusion | |
|---|---|---|---|---|---|---|---|---|
| | Difference | | Difference | | Difference | | Difference | |
| Time (min) | (μm) | p - Value | (μm) | p - Value | (μm) | p - Value | (μm) | p - Value |
| Baseline | 1.6 | 0.1082 | −0.1097 | 0.9602 | 2.278 | 0.5216 | 1.668 | 0.4121 |
| 0.0 | −0.873 | 0.6906 | 2.9 | 0.3984 | 1.028 | 0.7594 | 2.438 | 0.4494 |
| 2.5 | −0.1697 | 0.9291 | 3.54 | 0.467 | 4.178 | 0.2738 | 4.851 | 0.1419 |
| 5.0 | 0.9003 | 0.6815 | 3.504 | 0.4609 | 35.778 | 0.3037 | 11.558 | 0.2365 |
| 7.5 | 1.71 | 0.4602 | 8.749 | 0.2178 | 61.478 | 0.3645 | 22.838 | 0.2976 |
| 10.0 | 15.97 | 0.23 | 117.44 | 0.3073 | 127.54 | 0.3659 | 14.408 | 0.0968 |
| 12.5 | 129.43 | 0.3585 | 8.523 | 0.3171 | 128.47 | 0.3628 | 44.191 | 0.2819 |
| 15.0 | 227.64 | 0.1335 | 124.06 | 0.2767 | 142.11 | 0.3084 | 49.278 | 0.2828 |
| 17.5 | 0.087 | 0.972 | 5.13 | 0.0507 | 3.818 | 0.2926 | 8.538 | 0.1272 |
| 20.0 | 1.34 | 0.4538 | 1.874 | 0.3126 | 37.738 | 0.3894 | 5.448 | 0.0469 |
| 22.5 | 0.2703 | 0.8936 | 1.93 | 0.4903 | 123.03 | 0.3853 | 8.418 | 0.1888 |

As discussed hereinabove, comparing within subject groups (symptomatic or control), there was not a significant difference between provocations at each time. Likewise, there is not a significant difference when comparing mean thresholds between groups for each provocation at each time, potentially because of the small sample size and large variance in symptomatic data. However, there does appear to be a difference between tests when comparing symptomatic mean mechanosensory thresholds to control mechanosensory thresholds for each test (see Table 5). For instance, at the 5 minute measurement, the mean difference between symptomatic and control mechanosensory threshold (symptomatic mechanosensory threshold minus control mechanosensory threshold) is 0.9003 μm for provocation A; 3.504 μm for provocation B; 35.778 μm for provocation C; and 11.558 μm for provocation D. This indicates that adding risk factors to flexion causes a greater separation between symptomatic and control data. The tactile threshold of all subjects gradually increases during provocation, but the increase is greater for symptomatic subjects. The most effective risk factor in compromising tactile sensitivity in the fingertips may be wrist flexion. An increase in the symptomatic sample size will allow these to be statistically demonstrated.

A noteworthy trend occurred during recovery. The 17.5 minute interval data show a decrease in mechanosensory threshold from the 15 minute mechanosensory threshold, though not statistically significant. However, the 20 minute and 22.5 minute data show a trend of increasing mechanosensory thresholds, and the mean mechanosensory thresholds at these times are significantly different from baseline in each test for the control group and for provocation A at 22.5 minutes, B at 20 minutes, and D at 20 minutes for the symptomatic group. This may be caused by reactive hyperemia, and may have importance when considering work/rest cycles.

Subjects may also be tested using these provocations for diagnosis or evaluation of diabetic neuropathy. The ergonomic risk factors of the described provocations may affect subjects suffering from diabetic neuropathy differently.

Wrist extension has been shown to have greater effect on carpal tunnel pressure than flexion, but may not be as effective at provoking symptoms of CTS as flexion. To understand the effect of wrist posture on the median nerve, wrist extension may also be used, though this posture will require a different vibrometer configuration.

In one embodiment, the present invention includes three major elements: monitoring means, stimulation means, and provocation means. It is currently preferred that the aforementioned elements are interconnected with a computer for real-time monitoring and control as a “system” according to the invention. The apparatus may additionally be configured to be portable.

Monitoring involves measuring median nerve function before, during, and after provocative procedures. In currently preferred embodiments, the monitoring means include (but are not limited to): threshold monitoring means such as an event button that the subject hits when he or she feels the stimulus (exceeds threshold) (like a hearing test); electrophysiological monitoring means such as means to measure nerve conduction velocity and/or nerve compound action potential size; visual analog scale means by which the subject estimates the amount of sensation by sliding a bar, etc., where one end represents no sensation and the other end of the range the maximum sensation could imagine experiencing; means for monitoring skin temperature, which may change in some patient populations; means for monitoring blood flow to the wrist, hand, and fingers, which may change during a procedure according to the invention; and means for monitoring skin resistance, which may change.

Stimulation involves applying a sensory stimulation to the subject to ascertain the subject's threshold for perceiving the stimulation. In currently preferred embodiments, the stimulation means include (but are not limited to): a vibrometry probe, as discussed above; a means of applying thermal stimuli, such as cold pain, cooling, warming, heat pain, each of which measures threshold for a different type of sensory neuron and hence can potentially help in diagnosis; a means for applying suprathreshold stimulation, i.e., the amount of sensation evoked when any of the above stimuli exceed threshold (this links to the amplitude monitoring means discussed above (visual analog scale)); a means for applying electrical current to the skin can also be used to evoke sensation (the frequency of stimulation may be changed, as some investigators suggest it will select different nerve fiber populations by the frequency); and means for applying electrical voltage to the subject to activate the nerve for conduction velocity measurement.

Provocation means involve applying provocation to the wrist while it is held in flexion, from which the changes in the data obtained from stimulation and monitoring produce differences in patient populations. In currently preferred embodiments, the provocation means include (but are not limited to): direct pressure (instrument is shaped to the carpal tunnel and contains a force transducer so that force is monitored by the computer); tendon loading (devices are reduced to practice for monitoring the force applied to the fingers, means of attachment, vectors of finger displacement, perhaps the torque applied to finger tendons, the wrist angle might be monitored to estimate tangential forces on the median nerve in the carpal tunnel); a video system to measure angles and lengths of wrist, finger segments, etc.; and a pressure cuff for applying occlusion pressure, which is monitored by the computer.

The present invention may provide a total diagnostic protocol that is quick, automated, easy to learn, and can be applied to screening worker populations. The sensory evaluation protocol used in the present invention may be computerized. Stimuli may be generated in a double-blind, randomized format, and automated calibration checking may be used. Computerization allows the potential for implementation of modern psychophysical techniques for detection of false positive responses.

Although the present invention has been described with respect to the illustrated embodiments, various additions, deletions and modifications are contemplated as being within its scope. The scope of the invention is, therefore, indicated by the ensuing claims, rather than the foregoing description. All changes that come within the meaning and range of equivalency of the claims are to be embraced within their scope.

What is claimed is:

1. An apparatus for determining whether or not a subject suffers from a peripheral neuropathy, the apparatus comprising:
a stimulation element configured for applying a sensory stimulation to an area of the subject's body having a nerve;
a monitoring element in communication with the stimulating element, configured for measuring a function of the nerve; and
a provocation element that enhances alterations in the nerve's function.

2. The apparatus of claim 1, further comprising a computer in communication with the monitoring element and the provocation element.

3. The apparatus of claim 1, wherein the stimulation element comprises a vibrometry probe.

4. The apparatus of claim 3, wherein the monitoring element comprises an event button.

5. The apparatus of claim 1, wherein the stimulation element comprises a plurality of stimulating electrodes located over a plurality of digital nerves on a vertical plane of at least one finger.

6. The apparatus of claim 5, wherein the monitoring element comprises a recording electrode oriented for positioning over the median nerve proximal to the subject's wrist.

7. The apparatus of claim 1, wherein the provocation element comprises:
a flexion element for inducing a flexion angle in a wrist of the subject;
a surface configured for applying a compressive force to the wrist of the subject; and
a sensing element being capable of detecting the magnitude of the compressive force.

8. The apparatus of claim 7, wherein the surface comprises a round and compliant surface.

9. The apparatus of claim 8, wherein the sensing element comprises a pressure transducer.

10. The apparatus of claim 7, wherein the flexion angle comprises a substantially maximum degree wrist flexion of the subject.

11. The apparatus of claim 7, further comprising a goniometer for recording the flexion angle at a plurality of discrete times during the provocation.

12. The apparatus of claim 1, wherein the provocation element comprises:
a flexion element for inducing a flexion angle in a wrist of the subject;
a venous occlusion element for altering perfusion to the subject's wrist and the hand; and
a sensing element for detecting the magnitude of altered perfusion.

13. The apparatus of claim 12, wherein the sensing element comprises a pressure gauge.

14. The apparatus of claim 12, wherein the venous occlusion element comprises a pressure cuff.

15. The apparatus of claim 12, further comprising a goniometer for recording the flexion angle at a plurality of discrete times during the provocation.

16. The apparatus of claim 1, wherein the provocation element comprises:
a flexion element being capable of inducing a flexion angle in a wrist of the subject;
a loading element being capable of measuring an increase in tension on the tendons of at least one finger of the subject.

17. The apparatus of claim 16, wherein the loading element comprises:
at least one loop placed about at least one finger on the hand to be tested of the subject; and
a sensing element connected to the loop.

18. The apparatus of claim 17, wherein a system of strings and pulleys connect the sensing element to the loop.

19. The apparatus of claim 16, wherein the loading element comprises a load cell.

20. The apparatus of claim 1, wherein the stimulation element comprises a plurality of stimulating electrodes located over a plurality of digital nerves on a vertical plane of at least one finger, the monitoring element comprises a recording electrode oriented for positioning over the median nerve proximal to the subject's wrist, the provocation element comprises: a flexion element for inducing a flexion angle of substantially maximum degree wrist flexion of the subject in a wrist of the subject; a round and compliant surface configured for applying a compressive force to the wrist of the subject; and a sensing element comprising a pressure transducer being capable of detecting the magnitude of the compressive force, the apparatus further comprising:
a computer in communication with the monitoring element and the provocation element for calculating a baseline nerve function and a mean nerve function and timing stimulation element vibration amplitude and duration;
an additional stimulation element in communication with the computer comprising a vibrometry probe;
an additional monitoring element in communication with the computer comprising an event button;
a goniometer in communication with the computer for recording the flexion angle at a plurality of discrete times during the provocation;
a second provocation element in communication with the computer comprising:
a flexion element for inducing a flexion angle in a wrist of the subject;
a venous occlusion element comprising a pressure cuff for altering perfusion to the subject's wrist and the hand; and
a sensing element comprising a pressure gauge for detecting the magnitude of altered perfusion;
a third provocation element in communication with the computer comprising a flexion element being capable of inducing a flexion angle in a wrist of the subject, and a loading element being capable of measuring an increase in tension on the tendons of at least one finger of the subject, the loading element comprising at least one loop placed about at least one finger on the hand to be tested of the subject, and a sensing element connected using a system of strings and pulleys to the loop.

21. A method for determining whether or not a subject suffers from a peripheral neuropathy, comprising:
establishing a control nerve function for a provocation, the control nerve function representing an asymptomatic population;
applying a provocation over a period of time to the subject to be tested;
monitoring the subject during the period of time the provocation is applied to establish a test nerve function for the provocation; and
comparing the control nerve function and the test nerve function.
